(19) United States
(12) Patent Application Publication
(10) Pub. No.: US 2019/0077874 A1
(43) Pub. Date: Mar. 14, 2019

(54) METHODS AND COMPOSITIONS FOR THE TREATMENT OF TUBEROUS SCLEROSIS

(71) Applicant: National Tuberous Sclerosis Association, Inc., Silver Spring, MD (US)

(72) Inventor: Daniela BRUNNER, Bronx, NY (US)

(21) Appl. No.: 16/129,917

(22) Filed: Sep. 13, 2018

Related U.S. Application Data
(60) Provisional application No. 62/557,830, filed on Sep. 13, 2017.

(51) Int. Cl. C07K 16/28; A61P 35/00; A61K 9/00

(52) U.S. Cl. CPC C07K 16/2896; A61P 35/00; A61K 2039/545; A61K 9/0019; A61K 9/0053

(57) ABSTRACT

The treatment or prophylaxis of Tuberous Sclerosis Complex (“TSC”) and conditions associated with TSC is disclosed. Treatments involve the administration of one or more TLR4 inhibitors to patients with TSC. Such administration may also treat or forestall conditions associated with TSC, including epilepsy and lymphangioleiomyomatosis.

### CROSS REFERENCE TO RELATED APPLICATIONS

This application claims priority under 35 U.S.C. 119(e) to Provisional Application No. 62/557,830, filed Sep. 13, 2017, the entire contents of which are hereby incorporated by reference.

### FIELD OF INVENTION

The present invention relates to the treatment or prophylaxis of TSC and conditions or symptoms associated with TSC. The treatment involves the administration of one or more TLR4 inhibitors to a patient in need thereof.

### BACKGROUND OF INVENTION

Tuberous Sclerosis Complex (“TSC”), which may also be referred to as “tuberous sclerosis,” is a genetic disorder that causes benign growths (e.g., tubers and tumors) to form in many different organs, primarily in the brain, eyes, heart, kidney, skin and lungs. The aspects of TSC that most strongly impact quality of life are generally associated with the brain: seizures, developmental delay, intellectual disability and autism. Additionally, cysts growth in the kidneys can lead to serious consequences including death.

There is a wide variety in the signs and severity of TSC which are dependent on where growths develop and how severely are manifested. The brain, skin, heart, and kidneys are commonly affected. TSC lesions occurring in the skin and kidney contain smooth muscle cells, endothelial cells, adipocytes, and large neuronal appearing cells. Despite this complex cellular architecture, kidney and other lesions in TSC appear to be clonal in nature, based on locality and loss of heterozygosity (LOH) analyses. In the brain, TSC produces both subependymal tubers that line the ventricular sacs and subcortical hamartomas which serve as foci for epileptic discharges. Tumors that grow in the brain can block the flow of cerebral spinal fluid in brain ventricles leading to behavioral changes, nausea, headaches or many other symptoms. In the heart, TSC based tumors are usually at their largest at birth and then decrease in size as the individual gets older. These heart tumors, called cardiac rhabdomyomas, can cause problems at birth if they block the flow of blood or cause severe arrhythmia. Moreover, TSC based tumors in the eyes can present problems if they grow and block too much of the retina. TSC based tumors in the kidney (i.e., renal angiomyolipomas) can become so large they eventually disrupt normal kidney function and/or begin to bleed internally.

Additionally, epilepsy is a condition commonly associated with TSC. Although not completely understood, the incidence of TSC-induced epilepsy is thought to relate to the neuropathological features of TSC, including cortical tubers and other dysgenetic features. Accordingly, almost 90% of children with TSC have seizures, making this disease a leading cause of genetic epilepsy. Most children with TSC have onset of seizures during the first year of life, and up to one third of children with TSC will develop infantile spasms (“IS”). Almost all seizure types can be seen in a child with TSC, including tonic, clonic, tonic-clonic, atonic, myoclonic, atypical absence, partial, and complex partial. Seizures in TSC that appear generalized, both clinically and by electroencephalographic (EEG) characteristics, can have partial onset TSC and therefore might respond to anticonvulsant medications indicated for partial-onset seizures.

Typically, TSC based tubers are cortical malformations which may be detected in the brain. Tubers may not be actively growing in size. Epileptic foci tend to be located adjacent to tubers, but such adjacency is not always present. TSC may induce other growths categorized as tumors, which include subependymal nodules and subependymal giant cell astrocytomas.

Disease manifestations in different organ systems can vary widely between even closely related individuals. The protean nature of the condition can make clinical diagnosis challenging specifically when genetic testing is missing or inconclusive. A summary of diagnostic criteria for TSC may be found in Northrup, H. et al., Pediatr. Neurol. 49 (2013): 243-254, hereby incorporated by reference in its entirety. Clinical features of TSC are typically the means of diagnosis of the disease, while genetic testing to identify pathogenic mutations of the Tsc1 and/or Tsc2 gene may also be used for additional or secondary diagnosis of TSC. For example, a clinical diagnosis of TSC is “definite” when a subject suffers two major features (e.g., major associated conditions) of TSC (e.g., three or more hypomelanotic macules at least 5 mm in diameter, three or more angiofibromas and/or fibrous cephalic plaque, two or more ungual fibromas, shagreen patch, multiple retinal hamartomas, cortical dysplasias including tubers and cerebral white matter radial migration lines, subependymal nodules, subependymal giant cell astrocytoma, cardiac rhabdomyomas, lymphangioleiomyomatosis, more than two angiomyolipomas, etc.), or when a subject suffers one major feature with at least two minor features (e.g., minor associated condition) of TSC (e.g., confetti skin lesions, three or more dental enamel pits, more two or more intraorallibromas, retinal achromatic patch, renal cysts, nonrenal hamartomas, etc.). A clinical diagnosis of “possible” for having TSC may occur when a subject has either one major feature or two or more minor features associated with TSC. Additionally, the identification of a Tsc1 and/or Tsc2 pathogenic mutation is sufficient to make a definite diagnosis of TSC. However, other Tsc1 or Tsc2 variants which do not have as certain an effect on function are not sufficient to make a definite diagnosis of TSC. Moreover, approximately 15% of individuals with TSC have no mutation identified by conventional genetic testing, and a normal result does not exclude TSC or have any effect on the use of clinical diagnostic criteria to diagnose TSC.

Absent a diagnosis of TSC, some associated conditions of TSC may be prescribed standard medications unconnected to the etiology of the condition. For example, standard antiepileptic medications may provide relief for TSC-induced epilepsy, but are successful in only one third of patients due to different etiologies of the condition. Typically, antiepileptic medications treat the condition of epilepsy and not the underlying cause. Standard treatments of epilepsy include anticonvulsant medications, the vagus nerve stimulator, and the ketogenic diet. Since these medications are used to treat epilepsy with no distinction for the underlying etiological condition, many epileptic patients with TSC continue to have treatment-resistant seizures following medication.

The toll-like receptor 4 (TLR4) has been implicated in epilepsy but never before in TSC. Although brain inflammation is a major factor in the etiology of epilepsy, the impact of specific inflammatory mediators on neuronal excitability is not completely understood. However, in mice (C57BL/6) with acute and chronic seizures which were chemically induced by kainic acid and bicuculline, a proconvulsant pathway involving high-mobility group box-1 (HMGB1) proteins released from neurons and glia has been shown to interact with TLR4 See Maroso et al., Nature Medicine 16 (2010): 413-419, hereby incorporated by reference in its entirety. Inhibitors of HMGB1's action (e.g., competitors) and inhibitors of TLR4 retard seizure precipitation and decrease acute and chronic seizure recurrence. If analogous in humans, HMGB1-TLR4 signaling may contribute to generating and perpetuating seizures in humans. Of note, these results are limited to the kainic acid and bicuculline models of induced epileptic seizures. Here, an increase in HMGB1 causes increased activation of TLR4 and the subsequent inflammatory response. In contrast to these chemically induced models of epilepsy, the epilepsy of TSC often progresses to become intractable—meaning failure to be controlled with antiepileptic drugs that may affect, for example, the TLR4 pathway activity. Therefore, the pathology of seizures in TSC is not be well represented by standard chemically-induced models of epilepsy.

Due to significant distinctions in etiologies of epilepsy, TLR4 has not been implicated in TSC or TSC-associated conditions. Moreover, some receptors downstream of TLR4 may be shown to be upgregulated in some TSC tubers, but any connection to TLR4 is not known. For example, N-methyl D-aspartate receptor subtype 2B (“NR2B”) is upregulated in some human cortical tubers. However, this receptor is intimately involved in a number of important neuronal activities, and the upstream cause of NR2B upregulation in TSC has not been identified.

Additionally, antiepileptic medications for TSC-associated epilepsy suffer considerable drawbacks. For example, adrenocorticotropic hormone (ACTH) and vigabatrin (VGB) which are considered mainstay therapies for the treatment of seizures in TSC patients, are either not completely effective or not effective at all in some patients, and may have severe side effects. For example, VGB has been shown to be particularly effective in treating infantile spasms in the setting of TSC, but is ineffectual in more than 75% of patients with refractory partial-onset seizures. ACTH has demonstrated superior efficacy in comparison to steroids and is considered a first-line therapy for TSC-related seizures, but has serious side effects. In addition to stimulating the release of adrenal glucocorticoids, ACTH also suppresses the production of the stress-activated neuropeptide corticotropin-releasing hormone (CRH) through steroid-independent activation of central melanocortin receptors. Adverse effects occur in up to 85% of treated patients, most commonly being hypertension, immune suppression and infection, electrolyte imbalance, gastrointestinal disturbances, ocular opacities, hypertrophic cardiomyopathy, cerebral atrophy, growth impairment, hirsutism, irritability, and sleep disorders. Moreover, in patients with refractory partial-onset seizures, more than 75% may not benefit from VGB treatment.

Although some TSC patients benefit from existing medications despite their side effects, many are administered treatment-rounds of six or more antiepileptic medications in search of one that will be effective. As disclosed in French, J et al., The Lancet 388 (2016): 2153-63, hereby incorporated by reference in its entirety, close to 50% of all TSC patients are treatment resistant, having not responded to six or more previous antiepileptic drugs. The antiepileptic drugs that failed most frequently were levetiracetam in 67% patients, VGB in 66% patients, and topiramate in 58% patients.

Additionally, there is emerging evidence supporting the use of therapies which specifically target the inflammation pathways of TSC-growth since these pathways may be implicated in the proliferation of tuberous growth. TSC results from a mutation in the Tsc1 or the Tsc2 gene, both important regulators of cell growth and thus suppressing tuber formation and tumor growth. This regulation occurs through inhibition of the mammalian target of rapamycin (mTOR). mTOR is a protein kinase that controls cell growth, proliferation, and survival. The mTOR pathway not only plays a role in regulating cell growth and size, but it is also involved in the regulation of inflammatory mediators. Thus, the cellular components of the TSC-associated lesions could, in addition, contribute to the inflammatory response, as a result of Tsc1 or Tsc2 inactivation and deregulation of mTOR signaling. Defects in either the Tsc1 or Tsc2 gene leads to uncontrolled TSC tumor cell growth and proliferation via the dysregulated mTOR pathway. Accordingly, TSC induced tubers have a complex and sustained inflammatory phenotype due to the regulatory role of the mTOR complex, which includes both the innate and the adaptive immune response. Moreover, the inflammatory reaction involves the activation of the complement cascade and induction of the interleukin 1β (IL-1β) pro-inflammatory pathway.

In TSC patients, mTOR inhibitors have shown promise in providing regulation of the mTOR pathway that is absent when Tsc1 and/or Tsc2 genes are mutated, therefore preventing cell growth and proliferation. Specifically, mTOR inhibitors have been used the treatment of lymphangioleiomyomatosis (“LAM”), tumors due to TSC such as subependymal giant-call astrocytomas (“SEGAs”), skin and kidney tumors, and TSC-induced epilepsy. For example, case studies of TSC patients with medically refractory epilepsy and SEGAs demonstrated dramatic reduction of seizure frequency and SEGA size with the administration of mTOR inhibitors rapamycin or everolimus. Moreover, the effect of the mTOR inhibitor rapamycin over the course of treatment in a conditionally inactivated Tsc1 knockout (KO) mouse model has been shown to correlate with a significant decrease in seizure development. As shown in Zhang, et al., Neurobiology of Disease 80 (2015): 70-79, hereby incorporated by reference in its entirety, rapamycin or epicatechin-3-gallate (“ECG”) treatment of a TSC mouse model is able to reverse the increased production of two proteins implicated in the inflammatory pathway (IL-1β and CXCL10). This treatment was associated with reduced seizures and improved survival in the TSC mouse model. Treatment with mTOR inhibitors decreases expression of inflammatory mediators, before epilepsy onset in vivo, indicating that these mechanisms are downstream from mTOR. Therefore, expression of these inflammatory markers is not secondary to seizure onset, but possibly associated to the pathological mechanisms underlying TSC.

However, the use of mTOR inhibitors for these conditions of TSC also has its drawbacks. Rapamycin has been shown to inhibit both innate and adaptive immunological functions of cells apart from a direct action on the cell cycle. Therefore, adverse effects of mTOR inhibitors include immunosuppression, gastritis, mouth ulceration, pyrexia, pneumonia, lung or breathing problems, infections (including sepsis), and kidney failure, which can lead to death. In order to avoid the adverse effects of these mTOR inhibitors, treatments involving mTOR inhibitors require small doses over long treatment periods, which therefore could decrease the potential benefits of decreasing this inflammation pathway.

Other anti-inflammatory drugs also are also potentially problematic for use in the treatment of TSC. For example, aspirin, a nonsteroidal anti-inflammatory drug (“NSAID”), has been used for over a century to quell inflammation, leading to a body of evidence highlighting its potential in cancer prevention. Aspirin suppresses cell proliferation in a time- and dose-dependent manner through induction of the cyclin-dependent kinase inhibitor p21, reduced expression of cyclin-dependent kinase 4 and cyclin D3, and suppression of mTOR downstream signaling (i.e., reduced phosphorylation of the mTOR substrates 4E binding protein 1, serine/threonine kinase P70S6K and S6 ribosomal protein and inhibited glycogen synthase kinase 3 activity). Aspirin's effects suggest that the potential of anti-oncogenic effect obtained through anti-inflammatory therapy may well extend to TSC tumor manifestations which are dependent on mTOR over-activation. However, aspirin is contraindicated in TSC patients because of its adverse effects that include compromised platelet function, which may cause or increase bleeding in these already compromised patients. See Bissler, J, et al., American Journal of Kidney Diseases 39 (2002): 966-971. Other methods for suppression of inflammation in TSC are necessary for the treatment or prophylaxis of the disease and any conditions of the disease.

It is an object of the present invention to provide therapeutic agents, pharmaceutical compositions and methods treating TSC and the associated conditions caused by TSC.

### SUMMARY

In accordance with these objectives and others, provided herein are therapeutic active agents, pharmaceutical compositions and methods of treating TSC comprises administering a therapeutically effective amount of a TLR4 inhibitor to a patient diagnosed with TSC. The pharmaceutical compositions described herein comprise a therapeutically effective amount of a TLR4 inhibitor in combination with a pharmaceutically acceptable carrier, excipient or diluent. The methods may treat or cause the prophylaxis of an associated condition caused by TSC caused by administering a therapeutically effective amount of a TLR4 inhibitor. As TSC is often diagnosed prenatally or during the early post-natal period, it is also of interest to consider prophylactic treatment of TSC-associated conditions with a TLR4 inhibitor.

Without wishing to be bound by any theory, it is believed that inhibition of the TLR4 inflammatory pathway prevents or retards tuber formation and/or tumor growth in TSC. It is also believed that normalization of inflammatory pathway activity through TLR4 inhibition may normalize the activity in cells and cell-circuits that otherwise cause TSC-associated symptoms. Abnormal function may by secondarily related or independent of TSC-associated growth formation and activity. Active agents that antagonize toll-like receptor 4 (TLR4) signaling are capable of blocking inflammatory processes of TSC tubers and therefore are contemplated to be useful in retarding tuberous growth, and/or normalizing cell cycle and function. By antagonizing TLR4 in Tsc1 and/or Tsc2 deficient cells, organs, and/or circuits, cell size and cell proliferation can be mitigated prior and during tumor growth. Accordingly, the use of TLR4 inhibitors may result in a pharmaceutical effect with potentially less intractability than other medications. This disclosure relates to active agents and methods of the treatment or prophylaxis of TSC and/or the manifestations of TSC, including tumors, seizures, cognitive dysfunction, behavioral dysfunction, autism, and associated symptoms. Methods of treatment or the prophylaxis of TSC are provided comprising administering to a subject a pharmaceutical composition comprising a pharmaceutically acceptable excipient and a therapeutically effective amount of one or more TLR4 inhibitors or pharmaceutically acceptable salts, or prodrugs thereof.

These and other aspects of the invention will be apparent to those skilled in the art from the following detailed description, which is simply, by way of illustration, various modes contemplated for carrying out the invention. As will be realized, the invention is capable of additional, different, obvious aspects, all without departing from the invention. Accordingly, the Figures and specification are illustrative in nature and not restrictive.

### BRIEF DESCRIPTION OF FIGURES

FIG. 1 illustrates downstream inflammatory markers implicated in the activation of the canonical TLR4 pathway. Activation of TLR4 to illicit immune responses (e.g. with the Bacillus Calmette-Guérin (BCG) vaccine) is known to upregulate immunosuppressive cytokines IL-10, TGF-β, CCL2, and CCL5; angiogenic mediators VEGF, EGF and TGF-β; proinflammatory cytokines TNF, IL-1β, IL-6, and IL-8; and proteins related to the proliferation, apoptosis, inhibition and metastasis of tumor cells such as MAPK and NF-κB. Additionally, NR2B, a glutamate receptor involved in synaptic plasticity and neuronal excitability, is regulated downstream of IL-1β. It is known that NR2B is upregulated in TSC human cortical tubers as disclosed in Talos, D. et al., Ann. Neurol. 63 (2008): 454-465. Normalization of the TLR4 pathway could thus normalize NR2B activity, which could effectively suppress glutamate-induced hyperexcitability and seizures in TSC and animal models of TSC. The boxed proteins are shown to be upregulated in Tsc1 homozygous knockout mice as demonstrated in Example 1 and FIGS. 2A-2H.

FIG. 2 shows the upregulation of the inflammatory pathway receptor TLR4 in mice with TSC. In this mouse model, none, one, or both copies of the Tsc1 gene are foxed which allows for targeted excision in astrocytes by the CRE enzyme. Control mice had both copies of the Tsc1 gene (GFAP⁻; Cre⁻, Tsc^{flox/+}), heterozygous mice had only one Tsc1 copy (GFAP⁻ CRE⁻; Tsc1^{1/+}) and homozygous mice have both Tsc1 genes foxed (GFAP⁻ CRE⁺; Tsc1^{flox/flox}). Deletion of the two copies of Tsc1 in astrocytes leads to upregulation of TLR4 and leads to upregulation of several inflammatory mediators, many of which are downstream of TLR4 (see FIGS. 3-9). Control and heterozygous mice had similar expression of the receptor. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 3 shows the upregulation of the inflammatory protein marker VEGF-D in a homozygous knockout mouse model of TSC. VEGF-D, in particular, could be a specific culprit in LAM, exemplifying a particular beneficial effect of TLR4 inhibition. Control and heterozygous mice had similar expression of the marker. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 4 shows the upregulation of the VEGF-D receptor VEGF-R2, which mediates cellular responses to VEGF-D, in a homozygous knockout mouse model of TSC. Control and heterozygous mice had similar expression of the receptor. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 5 shows the upregulation of the inflammatory factor IL-1β in a homozygous knockout mouse model of TSC. Control and heterozygous mice had similar expression of the inflammatory factor. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 6 shows the upregulation of the inflammatory factor TNF-α in a homozygous mouse model of TSC. Control and heterozygous mice had similar expression of the inflammatory factor. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 7 shows the upregulation of the inflammatory factor IBA1 in a homozygous mouse model of TSC. Control and heterozygous mice had similar modulation. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 8 shows the upregulation of the inflammatory factor COX-2 in a homozygous mouse model of TSC. Control and heterozygous mice had similar modulation. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 9 shows the upregulation of the proliferation marker CD68 in a homozygous mouse model of TSC. Control and heterozygous mice had similar modulation. Data was analyzed with a one-way ANOVA, followed by Tukey's Multiple Comparisons Test. Levels of significance considered were p<0.05 (indicated by “*”), p<0.01 (indicated by “**”), p<0.001 (indicated by “***”), and p<0.0001 (indicated by “****”).

FIG. 10 shows the expression of TLR4 in a homozygous knockout mouse model of TSC after undergoing EEG electrode surgery and daily treatment with vehicle alone for four weeks, short rapamycin treatment (2 weeks of vehicle alone followed by 2 weeks of rapamycin), or long rapamycin treatment (4 weeks of treatment with rapamycin). As can be seen treatment did not confer differential expression to mice treated with rapamycin as compared to mice treated with vehicle alone. Data was analyzed with a one-way ANOVA, followed by Tukey's Multiple Comparisons Test. Levels of significance considered were p<0.05 or less and “ns” indicates a non statistical differential expression where indicated. Levels illustrated were normalized to those of knockout mice treated with vehicle.

FIG. 11 shows the expression of VEGF-D in a homozygous knockout mouse model of TSC after undergoing EEG electrode surgery and daily treatment with vehicle alone for four weeks, short rapamycin treatment (2 weeks of vehicle alone followed by 2 weeks of rapamycin), or long rapamycin treatment (4 weeks of treatment with rapamycin). As can be seen treatment did not confer differential expression to mice treated with rapamycin as compared to mice treated with vehicle alone. Data was analyzed with a one-way ANOVA, followed by Tukey's Multiple Comparisons Test. Levels of significance considered were p<0.05 or less. Levels illustrated were normalized to those of knockout mice treated with vehicle.

FIG. 12 shows the expression of IL-1β in a homozygous knockout mouse model of TSC after undergoing EEG electrode surgery and daily treatment with vehicle alone for four weeks, short rapamycin treatment (2 weeks of vehicle alone followed by 2 weeks of rapamycin), or long rapamycin treatment (4 weeks of treatment with rapamycin). As can be seen treatment did not confer differential expression to mice treated with rapamycin as compared to mice treated with vehicle alone. Data was analyzed with a one-way ANOVA, followed by Tukey's Multiple Comparisons Test. Levels of significance considered were p<0.05 or less. Levels illustrated were normalized to those of knockout mice treated with vehicle.

FIG. 13 shows the expression levels of TNF-α in a homozygous knockout mouse model of TSC after undergoing EEG electrode surgery and daily treatment with vehicle alone for four weeks, short rapamycin treatment (2 weeks of vehicle alone followed by 2 weeks of rapamycin), or long rapamycin treatment (4 weeks of treatment with rapamycin). As can be seen treatment did not confer differential expression to mice treated with rapamycin as compared to mice treated with vehicle alone. Data was analyzed with a one-way ANOVA, followed by Tukey's Multiple Comparisons Test. Levels of significance considered were p<0.05 or less. Levels illustrated were normalized to those of knockout mice treated with vehicle.

FIG. 14 shows the expression of IBA1 in a homozygous knockout mouse model of TSC after undergoing EEG electrode surgery and daily treatment with vehicle alone for four weeks, short rapamycin treatment (2 weeks of vehicle alone followed by 2 weeks of rapamycin), or long rapamycin treatment (4 weeks of treatment with rapamycin). As can be seen treatment did not confer differential expression to mice treated with rapamycin as compared to mice treated with vehicle alone. Data was analyzed with a one-way ANOVA, followed by Tukey's Multiple Comparisons Test. Levels of significance considered were p<0.05 or less. Levels illustrated were normalized to those of knockout mice treated with vehicle.

### DETAILED DESCRIPTION

In one aspect, the present disclosure provides pharmaceutical compositions comprising a therapeutically effective amount of a TLR4 inhibitor (e.g., a TLR4 antagonist, etc.). These pharmaceutical compositions may be used for the treatment or prophylaxis of TSC and/or the manifestations of TSC. In some embodiments, a method of treating a tumor associated with TSC is provided including effecting a regression in tumor size and/or slowing the rate of tumor growth by the administration of a TLR4 inhibitor. In some embodiments, a method of treating a tuber associated with TSC is provided including effecting a regression in tuber size and/or slowing the rate of tuber growth by the administration of a TLR4 inhibitor. In some embodiments, a method of treating a cell and/or cell circuit is provided including effecting a normalization (e.g., bring closer to the normal condition of a cell without a mutation associated with TSC) the cell and/or cell circuit by contact of a cell with a TLR4 inhibitor. In some embodiments, the normal condition of a cell may be defined as a cell without a homozygous Tsc1 mutation, without a heterozygous Tsc1 mutation, without a homozygous Tsc2 mutation, and/or without a heterozygous Tsc2 mutation. In some embodiments, a method of treating an organ dysfunction is also provided including effecting a normalization of the organ dysfunction by contact of the organ with a TLR4 inhibitor.

For convenience, certain terms employed in the specification, including the examples and appended claims, are collected here. Unless defined otherwise, all technical and scientific terms used herein have the same meaning as commonly understood by one of ordinary skill in the art to which this disclosure pertains.

Unless otherwise explicitly defined, the following terms and phrases are intended to have the following meanings throughout this disclosure:

All percentages given herein refer to the weight percentages of a particular component relative to the entire composition, including the carrier, unless otherwise indicated. It will be understood that the sum of all weight % of individual components within a composition will not exceed 100%.

The terms “a” or “an,” as used in herein means one or more. As used herein, the term “consisting essentially of” is intended to limit the invention to the specified materials or steps and those that do not materially affect the basic and novel characteristics of the claimed invention, as understood from a reading of this specification. The term “physiologically compatible” means that the component is generally regarded as safe and non-toxic for contact with human tissues at the levels employed.

The phrase “individual in need thereof” or “patient in need thereof” or “subject in need thereof” denotes an individual having TSC. In some implementations, the individual in need thereof is a patient that has been diagnosed with TSC. In other embodiments, the patient has one or more symptoms, manifestations, and/or conditions associated with TSC but has not been diagnosed with TSC (e.g., the individual has TSC, but has not been identified as having TSC, etc.). The term “prevent” or “prophylaxis” as used herein, includes delaying the onset of or progression of a disease or physiological manifestation of disease. The term “treat” includes reducing, diminishing, eliminating, ameliorating, forestalling, slowing the progression of, and/or delaying the onset of a given disease or physiological manifestation thereof. Non-limiting examples include humans, other mammals, bovines, rats, mice, dogs, monkeys, goat, sheep, cows, deer, and other non-mammalian animals. In some embodiments, a patient is human.

Unless otherwise specified, any compound disclosed herein which has one or more chiral centers may be in the form of a racemic mixture with respect to each chiral center, or may exist as pure or substantially pure (e.g., greater than about 98% enantiomeric excess (“ee”)) R or S enantiomers with respect to each chiral center, or may exist as mixtures of R and S enantiomers with respect to each chiral center, wherein the mixture comprises an enantiomeric excess of one or the other configurations, for example an enantiomeric excess (of R or 5) of more than 60% or more than 70% or more than 80% or more than 90%, or more than 95%, or more than 98%, or more than 99%. In some embodiments, any chiral center may be in the R or S configurations.

Any of the active ingredients (e.g., TLR4 inhibitors, mTOR inhibitors, NSAIDs, etc.) of the present disclosure may be in the form of pharmaceutically acceptable salts. “Pharmaceutically acceptable salts,” as used herein, denotes salts that are physiologically compatible, as defined herein, and that possess the desired pharmacological activity of the parent compound. Such salts include: acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate, and sodium hydroxide.

Any of the active agents described herein may be in the form of a “prodrug.” “Prodrug” as used herein refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor. A prodrug may be synthesized using reactants other than the corresponding drug. For example, prodrug of an active agent may be in the form of an in vivo hydrolysable ester of the specified active agent.

The term “signaling pathway” as used herein refers to a series of interactions between cellular and optionally extra-cellular components (e.g., proteins, nucleic acids, small molecules, ions, and lipids) that conveys a change in one component to one or more other components, which in turn may convey a change to additional components, which is optionally propagated to other signaling pathway components.

As defined herein, the terms “inhibition,” “inhibit,” “inhibiting,” and the like in reference to a protein-inhibitor interaction means negatively affecting (e.g., decreasing or diminishing) the activity or function of the protein (e.g., decreasing the signaling in the pathway stimulated by TLR4) relative to the activity or function of the protein in the absence of the inhibitor (e.g., TLR4 inhibitor). Inhibition includes, at least in part, partially or totally blocking stimulation, decreasing, preventing, or delaying activation, or inactivating, desensitizing, or down-regulating the signaling pathway or enzymatic activity or the amount of a protein (e.g., TLR4). As used herein, the term “inhibitor” refers to molecules or compounds that inhibit the action of a “native” or “natural” molecules or compounds (e.g., a receptor ligand). An inhibitor may be a compound that decreases the affinity of a receptor ligand for its substrate and/or a compound that decreases the downstream action of the receptor through direct or indirect action on the receptor. TLR4 antagonists, partial agonists, negative allosteric modulators, and other type of molecules that reduce the activation or consequences of the TLR4 activation by direct or indirect action on the TLR4 receptor may be referred to as TLR4 inhibitors herein.

In some embodiments, inhibition refers to inhibition of interactions of TLR4 which correlates with the production of downstream targets of the signaling pathway (e.g., VEGF, TNF, TGF, IL-1β, IL-6, IL-8, IL-10, CCL2, CCL5, MAPK, NF-κB, etc.). In some embodiments, the TLR4 inhibitor downregulates expression of TLR4. The TLR4 inhibitor may bind to the active site to effect a decrease in the activity of the TLR4 receptor (e.g., a TLR4 antagonist, etc.). For example, the TLR4 inhibitor may be an allosteric modulator which binds to a site distinct from that of orthosteric agonist binding site, yet is still capable of negatively regulating the activity or function of the receptor. In some embodiments, the TLR4 inhibitor may be an interfering RNA (e.g., mRNA, siRNA, shRNA etc.) capable of inhibiting gene expression by mediating RNA interference.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., protein-protein interaction, signaling pathway) of a protein (e.g., TLR4) in the absence of a compound as described herein.

The terms “treating” or “treatment” refers to any indicia of success in the treatment or amelioration of an injury, disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat epilepsy by decreasing the incidence of seizures and or causing remission of tuberous growth. In some embodiments of the compositions or methods described herein, treating TSC includes slowing the rate of growth or spread of tuberous cells, or reducing the growth of tumors. The term “treating” and conjugations thereof, include prevention of an injury, pathology, condition, or disease.

An “effective amount” is an amount sufficient for a compound to accomplish a stated purpose relative to the absence of the active ingredient (e.g., achieve the effect for which it is administered, treat a disease, reduce enzyme activity, increase enzyme activity, reduce signaling pathway, reduce one or more symptoms of a disease or condition, etc.). An example of an “effective amount” is an amount sufficient to contribute to the treatment, prevention, or reduction of a symptom or symptoms of a disease (e.g., TSC, etc.), which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins; each hereby incorporated by reference in their entirety).

“Disease” or “condition” refers to a state of being or health status of a patient or subject capable of being treated with the compounds or methods provided herein. In some embodiments, the disease is a disease related to (e.g., caused by) TSC.

The term “diagnose” refers to the act or process of identifying or determining the presence of a disease or condition (e.g., TSC, etc.) in a subject. Usually, a diagnosis of a disease or disorder is based on the evaluation of one or more clinical factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. “Diagnose” may refer to “definite” diagnosis of a disease or condition or a “possible” diagnosis of a disease or condition.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” covers placing the drug into the patient's body by any route of administration, including, without limitation, oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example mTOR inhibition treatment. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Typically, the pharmaceutical composition comprises an effective amount of a TLR4 inhibitor such as a TLR4 antagonist. Exemplary TLR4 inhibitors include, without limitation:

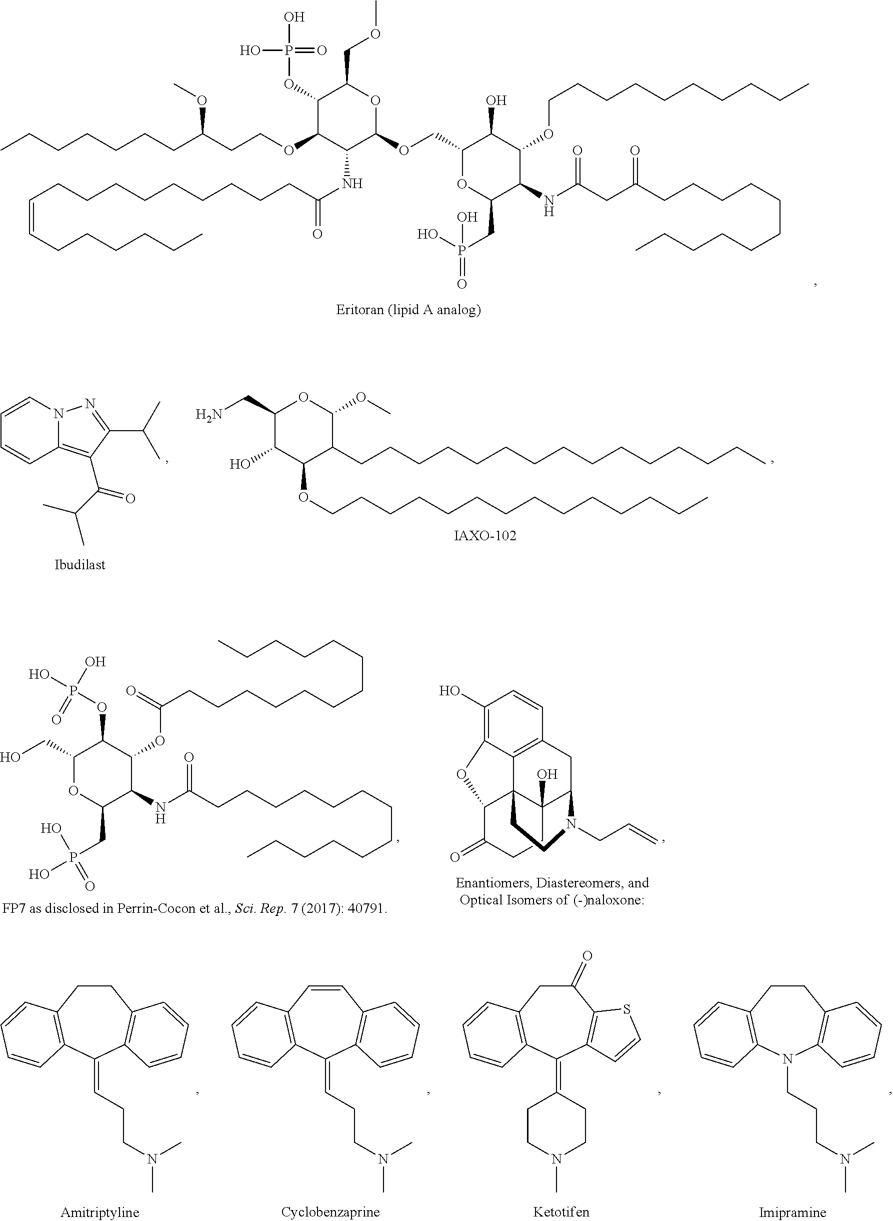

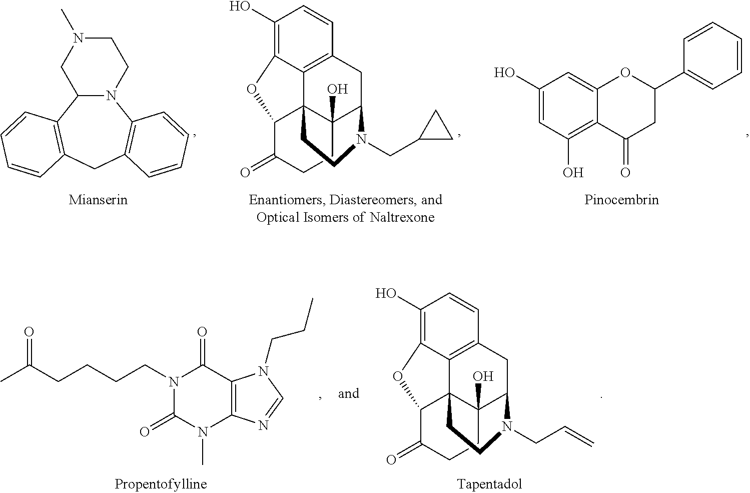

It will be understood that the TLR4 inhibitors described above are known in the art. The structures are provided for illustrative purposes. Any discrepancy between the structure and the known drug will be resolved in favor of the known drug.

Other TLR4 antagonists include lipopolysaccharides (“LPS”) from the photosynthetic bacterium Rhodobacter sphaeroides (“Lps-Rs”)37, or Bartonella quintana. The TLR4 antagonist may be a cyanobacterial lipopolysaccharide selected from an LPS of Schizothrix calcicola, Phormidium spp., Agmenellum quadruplicatum, Anabaena variabilis, Spirulina platensis, Anaystis nidulans, Microcystis aeruginosa, Anabaena flos-aquae. Any cyanobacterial lipolysaccharide disclosed in Molinaro, A, et al., Mar. Drugs 13 (2015): 4217-4230, hereby incorporated by reference in its entirety, may be used as a TLR4 antagonist. In some embodiments, the TLR4 antagonist may be a viral inhibitory peptide of TLR4 (from vaccinia virus protein A46) as disclosed in J. Immunol. 185 (2010): 4261-71, hereby incorporated by reference in its entirety. In some embodiments, the TLR4 inhibitor may inhibit the upregulation of TLR4. In some embodiments, the TLR4 inhibitor may be an anti-TLR4 antibody (e.g., a monoclonal antibody). In some embodiments, the TLR4 inhibitor may be an inverse agonist of TLR4, by which is meant that the inverse agonist is capable of binding to the same receptor as an agonist of TLR4, but the binding of the inverse agonist induces an antagonistic response. In some embodiments, the TLR4 inhibitor may be a negative allosteric modulator of TLR4.

In certain embodiments, the pharmaceutical composition comprises one or more TLR4 antagonists selected from Eritoran, Ibudilast, Rhodobacter sphaeroides LPS (Lps-Rs)37, IAXO-102, VIPER (from vaccinia virus protein A46), Cyanobacterial LPS (Cyp)38, Bartonella quintana lipopolysaccharide, natural and unnatural enantiomers of naloxone, amitriptyline, cyclobenzaprine, ketotifen, imipramine, mianserin, naltrexone, propentofylline,and tapentadol, or pharmaceutically acceptable salts, or prodrugs thereof. In some embodiments, the TLR4 inhibitor may be any TLR4 inhibitor in U.S. Pat. No. 9,072,760, hereby incorporated by reference, including selected from (2S-2-((4aR,6R,7R,8R,8aS)-7-acetamido-6-(2,3-bis(dodecyloxy)propoxy)-2,2-dimethylhexabydropyrano[3,2-d][1,3]dioxin-8-yloxy) propanoic acid, dodecyl 3,4,6-tri-O-acetyl-2-(acetylamino)-2-deoxy-beta-Dglucopyranoside, butyl 2-(acetylamino)-2-deoxy-3,4-di-O-methyl-beta-D-glucopyranoside, isopropyl 3,4,6-tri-O-acetyl-2-(acetylamino)-2-deoxyhexopyranoside, cyclohexyl-3,4,6-tri-O-acetyl-2-(acetylamino)-2-deoxy-alpha-D-glucopyranoside, hexyl 3,4,6-tri-O-acetyl-2-(acetylamino)-2-deoxy-beta-D-glucopyranoside, N-[(2R,3R,4R,5S,6R)-2-[(1′S,2′R,6′R,8′R,9′S)-dispiro [cyclohexane-1,4′-[3,5,7,10,12]pentaoxatricyclo[7.3.0.0-{2,6}]dodecane-11-'1′-cyclohexane]-8′ylmetboxy]-4,5-dihydroxy-6-(hydroxymethyl)oxan-3-yl]acetamide, (2R,3S,4R,5R,6R)-5-acetamido-2-(acetoxymethyl)-6-(((3aR,5R,5aS,8aS,8bR)-2,2,7,7-tetramethyl-tetrabydro-3aH-bis[1,3]dioxolo[4,5-b:4′5′-d]pyran-5-yl)methoxy)tetrabydro-2H-pyran-3,4-diyl-diacetate, N-((2R,3R,4R,5S,6R)-4,5-dihydroxy-6-(hydroxymethyl)-2-(((3 aR,5R,5 a S,8aS,8bR)-2,2,7,7-tetramethyltetrabydro-3 aH-bis[1,3]dioxolo[4,5-b:4′,5′-d]pyran-5-yl)methoxy)tetrahydro-2Hpyran-3-yl) acetamide, propyl 3,4,6-tri-O-acetyl-2-(acetylamino)-2-deoxyhexopyranoside, 1,3,4,6-tetra-O-acetyl-2-deoxy-2-(palmitoylamino)hexopyranose, 6-0-[2-(acetylamino)-2-deoxy-beta-D-glucopyranosyl]-3-0-isopentyl-1,2-0-(1-methyletbylidene)-alpha-D-xylo-hexofuranose, 6-0-[2-(acetylamino)-2-deoxy-beta-D-glucopyranosyl]-1,2-0-(1-methyletbylidene)-3-0-propyl-alpha-D-xylo-hexofuranose, 1,2-0-(1-methyletbylidene)-3-0-propyl-6-0-[3,4,6-tri-O-acetyl-2-(acetylamino)-2-deoxy-betaD-glucopyranosyl]-alpha-D-xylo-hexofuranose, 1,2-0-(1-methyletbylidene)-3-0-pentyl-6-0-[3,4,5-tri-O-acetyl-2-(acetylamino)-2-deoxy-betaD-glucopyranosyl]-alpha-D-xylo-hexofuranose, octyl 2-(acetylamino)-2-deoxy-beta-D-glycero-hexopyranoside, sec-butyl 2-(acetytamino)-2-deoxyhexopyranoside, sodium (2S,3S,4R,5R,6R)-3-((2S,3R,5S,6R)-3-acetamido-5-hydroxy-6-(hydroxymethyl)tetrabydro-2H-pyran-2-yloxy)-4,5,6-trihydroxytetrabydro-2H-pyran-2-carboxylate, 2-(acetylamino)-4-0-{2-(acetylamino)-4-0-[2-(acetylamino)-2-deoxybeta-D-glucopyranosyl}-2-deoxy-beta-D-glucopyranosyl-2-deoxy-D-glucopyranose, 3-acetamido-4, 5-dihydroxy-6-(hydroxymethyl)tetrabydro-2H-pyran-2-yl dihydrogen phosphate, sodium salt, sulfuric acid compound with (2R)-4-amino-N-{(1R,2S,3S,4R,5S)-5-amino-2-[(3-amino-3-deoxyalpha-D-glucopyranosyl)oxy]-4-[(6-amino-6-deoxy-alpha-Dglucopyranosyl)oxy]-3-hydroxycyclohexyl]-2-hydroxybutanamide (1:1), (4R)-4-((2S)-2-((2R)-2-((3R,4R,5S,6R)-3-acetamido-2,5-dihydroxy-6-(hydroxymetby 1)tetrabydro-2H-pyran-4-yloxy) propanamido)propanamido)-5-amino-5-oxopentanoic acid, Uridine 5′-diphospho-N-acetylglucosamine sodium salt, Uridine 5′-diphospho-N-acetylgalactosamine disodium salt, 2-(acetylamino)-3-0-{4-0-[2-(acetylamino)-2-deoxy-3-0-alpha-D-xylo-hexopyranuronosyl-betaD-ribo-hexopyranosyl]-beta-D-xylo-hexopyranuronosyl}-2-deoxy-Dglucopyranose, 2-(acetylamino)-2-deoxy-3-0-(6,8-dideoxy-beta-L-glycerooctopyranosyl-7-ulose)-4-0-sulfo-L-erythro-hexopyranose, 2-(acetylamino)-2-deoxy-4-0-hexopyranosylhexopyranose, 2N-{(1S,2S,3R)-1-[(beta-L-glycero-hexopyranosyloxy)methyl]-2,3-dihydroxyheptadecyl) hexacosanamide, dimethyl 5-(acetylamino)-3,5-dideoxy-D-erythro-non-2-ulopyranosidonate, methyl 2-(acetylamino)-2-deoxy-3-0-hexopyranosylhexopyranoside, 8-{[2-(acetylamino)-4-0-[2-(acetylamino)-2-deoxyhexopyranosyl]-2-deoxy-6-0-(6-deoxyhexopyranosyl) hexopyranosyl]oxy)octyl acetate, octyl 2-(acetylamino)-2-deoxyhexopyranoside, 2-(acetylamino)-2-deoxy-4-0-(6-deoxyhexopyranosyl)-3-0-hexopyranosylhexopyranose, 2-(acetylamino)-2-deoxy-alpha-D-lyxo-hexopyranose, 2-(acetylamino)-2-deoxy-D-glucopyranose, allyl 3,4,6-tri-O-acetyl-2-(acetylamino)-2-deoxy-beta-D-lyxohexopyranoside, 48N-{(1S,2R,3E)-1-[(beta-L-ribo-hexopyranosyloxy)methyl]-2-hydroxy-3-heptadeceny}octadecanamide, sodium ((3S,6R)-5-acetamido-3,4, 6-trihydroxytetrahydro-2H-pyran-2-yl)methyl phosphate, 2-((2R,5S)-3-acetamido-2,5-dihydroxy-6-(hydroxymethyl)tetrahydro-2H-pyran-4-yloxy)propanoic acid, allyl 2-(acetylamino)-2-deoxy-beta-D-glycero-hexopyranoside, 51 1,3,4,6-tetra-O-acetyl-2-(acetylamino)-2-deoxy-beta-D-glycerohexopyranose, 2-(acetylamino)-2-deoxy-beta-D-glycero-hexopyranose, 4-0-[2-(acetylamino)-2-deoxyhexopyranosyl]-1,5-anhydro-2-deoxyhexitol, ethyl 2-(acetylamino)-2-deoxy-beta-D-glycero-hexopyranoside, ethyl 3,4,6-tri-O-acetyl-2-(acetylamino)-2-deoxy-beta-D-glycerohexopyranoside, 5-acetamido-6-((1R,2R)-3-(3-(3-acetamido-5-hydroxy-6-(hydroxymethyl)-4-(3,4,5-trihydroxy-6-(hydroxymethyl)tetrahydro-2H>-pyran-2-yloxy) tetrahydro-2H-pyran-2-yloxy)-6-(4,5-dihydroxy-6-((E)-3-hydroxy-2-stearamidooctadec-4-e cyclohexanamine compound with 1,6-di-O-phosphono-beta-Dglycero-hexopyranose (4:1) hydrate, 4-0-(3-0-{2-(acety lamina)-2-deoxy-4-0-(6-deoxy hexopyranosyl)-3-0-[2-0-(6-deoxyhexopyranosyl)hexopyranosyl]hexopyranosyl} hexopyranosyl)hexopyranose, 3-0-(3-0-{2-(acetylamino)-2-deoxy-3-0-[2-0-(6-deoxyhexopyranosyl)hexopyranosyl] hexopyranosyl} hexopyranosyl)-D-arabinose, 2-(acetylamino)-2-deoxy-3-0-(6-deoxyhexopyranosyl)-4-0-hexopyranosylhexopyranose, nonyl 2-(acetylamino)-2-deoxy-beta-D-glycero-hexopyranoside, octadecyl 2-(acetylamino)-2-deoxy-beta-D-glycero-hexopyranoside, 4-0-{6-0-[5-(acetylamino)-3,5-dideoxy-D-erythro-non-2-ulopyranonosyl]hexopyranosyl}hexopyranose, 2-deoxy-2-(propionylamino)-D-glucopyranose, cyclohexane-1,2,3,4,5,6-hexayl hexakis(dihydrogen phosphate), magnesiun potassium salt, 1,3,4,6-tetra-O-acetyl-2-(acetylamino)-2-deoxy-beta-D-glucopyranose, 2-(acetylamino)-2-deoxy-D-galactopyranose hydrate, [(4R)-5-acetamido-3,4,6-triacetyloxy-oxan-2-yl]methyl acetate, [5-acetamido-3-acetyloxy-2-(acetyloxymethyl)-6-hexadecoxy-oxan-4-yl] acetate, (5-acetamido-3,4-diacetyloxy-6-pentoxy-oxan-2-yl)methyl acetate, (5-acetamido-3,4-diacetyloxy-6-methoxy-oxan-2-yl)methyl acetate, N-[2-ethoxy-4, 5-dihydroxy-6-(hydroxymethyl)oxan-3-yl]acetamide, [2,5-diacetyloxy-6-(acetyloxymethyl)-3-(dodecanoylamino)oxan-4-yl] acetate, or combinations thereof.

The compounds can be combined with one or more pharmaceutically acceptable excipients to form a pharmaceutical composition. Example excipients include diluent, carrier or filler. The compositions can be formulated for enteral, parenteral, topical, transdermal, or pulmonary administration. The compounds can be formulated for immediate release, controlled release, and combinations thereof. Examples of controlled release formulations include delayed release, extended release, pulsatile release, and combinations thereof.

The amount administered depends on the pharmaceutical formulation, route of administration, etc. and is generally empirically determined in routine trials, and variations will necessarily occur depending on the target, the host, the route of administration, etc. It is within the skill of the art to determine the therapeutically effective amounts for TLR4 inhibitor. Generally, the quantity of active TLR4 inhibitor (e.g., Eritoran, Ibudilast, Rhodobacter sphaeroides LPS (Lps-Rs)37, IAXO-102, VIPER (from vaccinia virus protein A46), Cyanobacterial LPS (Cyp)38, Bartonella quintana lipopolysaccharide, natural and unnatural enantiomers of naloxone, amitriptyline, cyclobenzaprine, ketotifen, imipramine, mianserin, naltrexone, propentofylline, tapentadol, etc.) in a unit dose of preparation may be varied or adjusted from about 1 μg, 100 μg, 0.5 mg, or 1 mg to about 30 mg, 100 mg, 300 mg, or 1000 mg, according to the particular application. In some embodiments, the effective amount of TLR4 inhibitor may be between about 1 μg and about 500 mg or from about 1 μg to about 50 mg or about 1 μg to about 70 mg or about 1 mg to about 1000 mg or about 1 mg to about 500 mg or about 1 μg to about 1 mg or about 50 to about 600 mg. In a particular embodiment, unit dosage forms are packaged in a multipack adapted for sequential use, such as blister pack, comprising sheets of at least 6-, 9-, or 12-unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

According to the present invention, methods of this invention comprise administering an effective amount of a composition of the present invention as described above to the subject. The effective amount of the composition, the use of which is in the scope of present invention, will vary somewhat from subject to subject, and will depend upon factors such as the age and condition of the subject and the route of delivery. Such dosages can be determined in accordance with routine pharmacological procedures known to those skilled in the art. For example, the compounds of the present invention can be administered to the subject in an amount ranging from a lower limit from about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2.0, 3.0, 4.0, 5.0, 6.0, 7.0, 8.0, 9.0, or 10% to an upper limit ranging from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% by weight of the composition. In some embodiments, the compounds comprise from about 0.05 to about 95% by weight of the composition. In other embodiments, the compounds comprise from about 0.05 to about 60% by weight of the composition. In still other embodiments, the compounds comprise from about 0.05 to about 10% by weight of the composition.

The TLR4 inhibitors and pharmaceutical compositions described herein may be used to treat a variety of diseases or conditions associated with TSC that are considered different manifestations of TSC including, but not limited to, brain injury and pathology, skin abnormalities, skin growths under or around the nails, facial lesions, developmental delays, intellectual disability, learning disabilities, hyperactivity, raging outbursts, aggression, repetitive behaviors, social and emotional withdrawal, communication and/or social interaction problems, autism spectrum disorder, communication disorders, social interaction disorders, kidney problems, heart problems, lung problems, pulmonary leiomyomas, eye abnormalities, retinal lesions, neuropathic pain, hypertension, autism, cognitive dysfunction, behavioral dysfunction, pancreatic neuroendocrine tumors, liver hamartomas, retinal hamartoma, sleep disorder, epilepsy, clonic seizures, tonic-clonic seizures (grand mal seizures), tonic seizures, akinetic seizures, atypical absence seizures, myoclonic seizures, complex partial seizures, generalized squires, status epilepticus, neuroinflammation after a seizure, infantile spam, endometriosis, cancer, inflammatory bowel disease, arthritis, rheumatoid arthritis, skin inflammation, vascular inflammation, kidney disease, heart disease, acne vulgaris, asthma, hypersensitivities, pelvic inflammatory disease, sarcoidosis, vasculitis, interstitial cystitis, autoimmune diseases, cortical tubers, subependymal nodules, subependymal giant-cell astrocytomas (SEGA), anxiety, renal angiomyolipomas, renal cysts, depression, aggression, sudden rage, attention deficit hyperactivity disorder, acting out, obsessive-compulsive disorder, repetitive behavior, destructive behavior, self-harming behavior, autism spectrum disorder, angiomyolipomas, renal cell carcinoma, oncocytomas, rhabdomyomas, phakomas, liver tumors, liver cysts, lung tumors, lung cysts, pancreas tumors, pancrease cysts, bone cysts, rectal polyps, gum fibromas, dental pits, hypomelanic macules, facial angiofibromas (adenoma sebaceum), forehead plaques, shagreen patches, ungual and/or subungual fibromas, molluscum fibrosum (skin tags), café au lait spots, poliosis, lymphangioleiomyomatosis (LAM) and/or its manifestations including breathing difficulty, chylous pleural effusions, and/or chylothorax, and multinodular multifocal pneumocyte hyperplasia (MMPH). In some embodiments, the TLR4 pharmaceutical compositions are administered prior to the manifestations of TSC in a preventative treatment modality. In some embodiments, the patient in need thereof does not suffer from epilepsy.

Methods for treatment the treatment of TSC and/or a TSC-associated condition in a patient in need thereof are provided comprising administering to the patient a therapeutically effective amount of a pharmaceutical composition comprising a TLR4 inhibitor. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.). In some embodiments, the pharmaceutical composition is coadministered with another active agent (e.g., rapamycin).

In some embodiments, a method is provided for the treatment or prophylaxis of epilepsy in a patient in need thereof which may comprise administering to said patient a therapeutically effective amount of a pharmaceutical composition comprising a TLR4 inhibitor. The epilepsy may comprise infantile spasms, and/or seizures selected from complex partial, partial, tonic, clonic, tonic-clonic, atonic, myoclonic, atypical absence, and combinations thereof. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of lymphangioleiomyomatosis and/or the manifestations thereof in a patient in need thereof is provided comprising administering to said patient a therapeutically effective amount of a pharmaceutical composition comprising a TLR4 inhibitor. The manifestations of lymphangioleimyomatosis may include breathing difficulties, chylous pleural effusions, chylothorax and combinations thereof. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of angiomyolipomas in a patient in need thereof is provided comprising administering to said patient a therapeutically effective amount of a pharmaceutical composition comprising a TLR4 inhibitor. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of skin manifestations of TSC in a patient in need thereof is provided comprising administering to said patient a therapeutically effective amount of a pharmaceutical composition comprising a TLR4 inhibitor. The skin manifestations of TSC may comprise angiofibromas, ungula fibromas, subungual fibromas, forehead plaques, and combinations thereof. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of subependymal nodules and/or subependymal giant cell astrocytomas in a patient in need thereof is provided comprising administering to said patient a therapeutically effective amount of a pharmaceutical composition comprising a TLR4 inhibitor. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of cardiac rhabdomyomas in a patient in need thereof is provided comprising administering to said patient a therapeutically effective amount of a pharmaceutical composition comprising a TLR4 inhibitor. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of retinal hamartoma in a patient in need thereof is provided comprising administering to said patient a therapeutically effective amount of a pharmaceutical composition comprising a TLR4 inhibitor. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of pancreatic neuroendocrine tumors in a patient in need thereof is provided comprising administering to said patient a therapeutically effective amount of a pharmaceutical composition comprising a TLR4 inhibitor. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis liver hamartomas in a patient in need thereof is provided comprising administering to said patient a therapeutically effective amount of a pharmaceutical composition comprising a TLR4 inhibitor. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of cysts in a patient in need thereof comprising administering to said patient a therapeutically effective amount of a pharmaceutical composition comprising a TLR4 inhibitor. In some embodiments, the cysts may be renal cysts. The patient may have TSC. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

In some embodiments, a method for the treatment or prophylaxis of autism in a patient in need thereof is provided comprising administering to said patient a therapeutically effective amount of a pharmaceutical composition comprising a TLR4 inhibitor. In some embodiments, the patient has been diagnosed with TSC. In other embodiments, the patient has not been diagnosed with TSC. In some embodiments, the patient has a definite diagnosis of TSC (e.g., two or more major associated conditions, one major associated condition and two or more minor associated conditions, etc.). In some embodiments, the patient has a possible diagnosis of TSC (e.g., one major associated condition, two or more minor associated conditions, etc.).

The method of treatment and/or prophylaxis may be combined with one or more drugs effective to treat TSC and/or conditions associated therewith. In some embodiments, method may treat one or more neoplasms associated with TSC. In some embodiments, a method for the treatment of epilepsy associated with TSC is provided comprising administering a therapeutically effective amount of a TLR4 inhibitor. In some embodiments, a method for the treatment of epilepsy associated with TSC is provided comprising administering a therapeutically effective amount of a TLR4 inhibitor and an mTOR inhibitor (e.g., rapamycin, everolimus, sirolimus, etc.). In some embodiments, a method for the treatment of epilepsy associated with TSC is provided comprising administering a therapeutically effective amount of a TLR4 inhibitor and VGB and/or ACTH and/or levetiracetam and/or topiramate. In some embodiments, a method for the treatment of lymphangioleiomyomatosis associated with TSC is provided comprising administering a therapeutically effective amount of a TLR4 inhibitor. In some embodiments, a method for the treatment of lymphangioleiomyomatosis associated with TSC is provided comprising administering a therapeutically effective amount of a TLR4 inhibitor and an mTOR inhibitor. In some embodiments, a method for the treatment of lymphangioleiomyomatosis associated with TSC is provided comprising administering a therapeutically effective amount of a TLR4 inhibitor and a VEGF inhibitor. In some embodiments, a method for the treatment of cardiac rhabdomyomas associated with TSC is provided comprising administering a therapeutically effective amount of a TLR4 inhibitor. In some embodiments, a method for the treatment of renal angiomyolipomas associated with TSC is provided comprising administering a therapeutically effective amount of a TLR4 inhibitor.

The conditions associated with TSC may manifest prior to a diagnosis of TSC in many patients. Since somatic mutations may occur in the Tsc1 and/or Tsc2 genes, a patient may be diagnosed with the conditions associated with TSC prior to a diagnosis of TSC itself. Therefore, a patient in need thereof may not have been diagnosed with TSC when the methods of treatment described herein are applied (for example, a patient may have lymphangioleiomyomatosis or angiomyolipomas or epilepsy or another TSC-associated condition, but that TSC is the cause of the TSC-associated condition is unknown at the time of treatment, etc.). In some embodiments, a method for the treatment of lymphangioleiomyomatosis in a patient in need thereof may comprise administration of an effective amount of a pharmaceutical composition comprising a TLR4 inhibitor prior to diagnosis of TSC in the patient. In some embodiments, a method for the treatment of angiomyolipomas in a patient in need thereof may comprise administration of an effective amount of a pharmaceutical composition comprising a TLR4 inhibitor prior to diagnosis of TSC in the patient.

Additionally, the patient may have been diagnosed with TSC and TSC-associated conditions have not yet manifested. In some embodiments, the patient has been diagnosed with TSC and TSC-associated conditions. In some embodiments, the patient may have received a “definite” diagnosis of TSC. In some embodiments, the patient may have received a “possible” diagnosis of TSC. In some embodiments, the patient may have the symptoms of one or more (e.g., one, two, three, four, etc.) associated conditions of TSC. In some embodiments, the method for the treatment or prophylaxis of TSC in a patient in need thereof comprises administration of an effective amount of a pharmaceutical composition comprising a TLR4 inhibitor, wherein the patient is a child under the age of 10 or 5 or 2. In some embodiments, the patient is an infant. In some embodiments, the patient is a newborn. In some embodiments, the patient is prenatal. In some embodiments, the patient has been diagnosed prenatally or during the early post-natal.

In certain embodiments, TLR4 inhibitors as described herein are administered alone or in combination with one or more additional active agents. Additional active agents can be administered simultaneously in the same dosage form or in separate dosage forms. Additional active agents comprise but are not limited to mTOR inhibitors, analgesics, anti-inflammatory drugs, anti-oncogenic drugs, angiogenesis inhibitors, antipyretics, antidepressants, antiepileptics, antihistamines, antimigraine drugs, antimuscarinics, anxioltyics, autophagy inhibitors, sedatives, hypnotics, antipsychotics, bronchodilators, anti-asthma drugs, cardiovascular drugs, corticosteroids, dopaminergics, electrolytes, gastro-intestinal drugs, G6PD inhibitors, muscle relaxants, nutritional agents, vitamins, parasympathomimetics, pro-apoptotic drugs, stimulants, anorectics and anti-narcoleptics. In some embodiments, and in particular when a patient has been diagnosed with epilepsy associated with TSC, TLR4 inhibitors are administered in combination with an mTOR inhibitor (e.g., rapamycin, everolimus, sirolimus). In some embodiments, TLR4 inhibitors are administered in combination with anti-epileptic drugs (e.g., adrenocorticotropic hormone, vigabatrin, etc.). “Adjunctive administration”, as used herein, means the TLR4 inhibitors can be administered in the same dosage form or in separate dosage forms with one or more other active agents. In some embodiments, the TLR4 inhibitor and additional active agent are in the form of a “co-drug,” by which is meant that the TLR4 inhibitor and additional active are covalently linked to one another via a labile bond, or ionically linked to one another to form a single working composition. For example, the pharmaceutical composition may comprise a therapeutically effective amount of a co-drug of a TLR4 inhibitor and an mTOR inhibitor (e.g., rapamycin). Such co-drug compositions may provide a controlled or sustained release of both the TLR4 inhibitor and additional active ingredient for a systemic or local pharmacologic or physiologic effect for organs affected by TSC including, for example, the brain, heart, lungs, and/or kidneys.

In some embodiments, the compounds described herein can be coadministered with one or more other therapeutic agents. In certain embodiments, the additional agents may be administered separately, as part of a multiple dose regimen, from the compounds of the present disclosure (e.g., sequentially, e.g., on different overlapping schedules with the administration of one or more TLR4 inhibitors). In still another embodiment, these agents can be given as a separate dose that is administered at about the same time that one or more TLR4 inhibitors are administered (e.g., simultaneously with the administration of one or more TLR4 inhibitors. When the compositions of the present disclosure include a combination of a TLR4 inhibitor and one or more additional therapeutic or prophylactic agents, both the compound and the additional agent can be present at dosage levels of between about 1 to 100%, and more preferably between about 5 to 95% of the dosage normally administered in a monotherapy regimen. In some embodiments, coadministration of a therapeutic regimen may involve the use of a codrug.

In other embodiments, these agents may be part of a single dosage form, mixed together with the compounds of the present disclosure in a single composition. For example, the pharmaceutical composition may comprise a therapeutically effective amount of a TLR4 inhibitor and therapeutically effective amount of a mTOR inhibitor (e.g., rapamycin, everolimus, sirolimus etc.). The pharmaceutical composition may comprise a therapeutically effective amount of a TLR4 inhibitor and one or more drugs effective to treat TSC and/or conditions associated therewith. In some embodiments, the pharmaceutical composition may comprise a therapeutically effective amount of a TLR4 inhibitor and therapeutically effective amount of an anti-epileptic medication (e.g., ACTH, VGB, levetiracetam, topiramate, etc.). In some embodiments, the pharmaceutical composition may comprise a therapeutically effective amount of a TLR4 inhibitor and therapeutically effective amount of a lymphangioleiomyomatosis medication (e.g., sirolimus, etc.). In some embodiments, the pharmaceutical composition may comprise a therapeutically effective amount of a TLR4 inhibitor and an NSAID (e.g., aspirin). It is within the skill of the art to determine the therapeutically effective amounts for active ingredients for co-administration as well. In some embodiments, the pharmaceutical composition may comprise a therapeutically effective amount of a TLR4 inhibitor and an amount of rapamycin from about 0.1 mg up to 50 mg, such as 0.1 mg to 10 mg. e.g. 0.1 mg, 0.25 mg, 0.5 mg, 0.75 mg, 1 mg, 2.5 mg, 5 mg, or 10 mg, more preferably from 0.5 mg to 10 mg. In some embodiments, the pharmaceutical composition may comprise a therapeutically effective amount of a TLR4 inhibitor and an amount of sirolimus from about 0.1 mg up to 15 mg, such as 0.1 mg to 10 mg. e.g. 0.1 mg, 0.25 mg, 0.5 mg, 0.75 mg, 1 mg, 2.5 mg, 5 mg, or 10 mg, more preferably from 0.5 mg to 10 mg. In some embodiments, the pharmaceutical composition may comprise a therapeutically effective amount of a TLR4 inhibitor and an amount of an anti-epileptic medication (e.g., ACTH, VGB, levetiracetam, topiramate, etc.) from about 0.1 mg up to 50 mg, such as 0.1 mg to 10 mg. e.g. 0.1 mg, 0.25 mg, 0.5 mg, 0.75 mg, 1 mg, 2.5 mg, 5 mg, or 10 mg, more preferably from 0.5 mg to 10 mg.

Specific examples of active compounds that can be co-administered (e.g., adjunctively administered, etc.) with the TLR4 inhibitors include, but are not limited to, aceclofenac, acetaminophen, acetazolamide, ACTH, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clobazam, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, eslicarbazepine acetate, estazolam, ethosuximide, etodolac, everolimus, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lacosamide, lamotrigine, lesopitron, levetiracetam, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methyl salicylate, methysergid(e), metoclopramide, mianserin, mifepristone, milnacipran, minaprine, mirtazapine, moclobemide, modafinil, molindone, morphine, morphine hydrochloride, nabumetone, nadolol, naproxen, naratriptan, nefazodone, neurontin, nitrazepam, nomifensine, nortriptyline, olanzapine, olsalazine, ondansetron, opipramol, orphenadrine, oxaflozane, oxaprazin, oxazepam, oxcarbazepine, oxitriptan, oxycodone, oxymorphone, pancrelipase, parecoxib, paroxetine, pemoline, pentazocine, pepsin, perampanel, perphenazine, phenacetin, phendimetrazine, phenmetrazine, phenobarbital, phenylbutazone, phenytoin, phosphatidylserine, pimozide, piracetam, pirlindole, piroxicam, pizotifen, pizotyline, pramipexole, prednisolone, prednisone, pregabalin, primidone, propanolol, propizepine, propoxyphene, protriptyline, quazepam, quinupramine, rapamycin, reboxitine, reserpine, retigabine, risperidone, ritanserin, rivastigmine, rizatriptan, rofecoxib, ropinirole, rotigotine, rufinamide, salsalate, sertraline, sibutramine, sildenafil, sodium valproate, stiripentol, sulfasalazine, sulindac, sumatriptan, tacrine, temazepam, tetrabenozine, thiazides, thioridazine, thiothixene, tiagabine, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, vigabatrin, viloxazine, vitamin E, zimeldine, ziprasidone, zolmitriptan, zolpidem, zonisamide, zopiclone and isomers, salts, and combinations thereof. In some embodiments, the one or more TLR4 inhibitors is coadministered with one or more mTOR inhibitors (e.g., rapamycin, etc.), VEGF modulators, IL-1β modulators, TNF-α modulators, TNF-β modulators, IBA1 modulators, COX-2 modulators, CD68 modulators, CCL2 modulators, CCL5 modulators, EGF modulators, TNF modulators, IL-6 modulators, IL-8 modulators, and combinations thereof. In some embodiments, the one or more TLR4 inhibitors is coadministered with modulators (e.g., antagonist, partial agonists, allosteric modulators, agonists, etc.) of MAPK and NF-κB. In preferred embodiments, the one or more TLR4 inhibitors is coadministered with one or more mTOR inhibitors (e.g., rapamycin, etc.), VEGF inhibitors, IL-1β inhibitors, NR2B inhibitors, TNF-α inhibitors, TNF-β inhibitors, IBA1 inhibitors, COX-2 inhibitors, CD68 inhibitors, CCL2 inhibitors, CCL5 inhibitors, EGF inhibitors, TNF inhibitors, IL-6 inhibitors, IL-8 inhibitors, and combinations thereof. Most preferably, the TLR4 inhibitor is coadministered with an mTOR inhibitor.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally, or by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg (e.g., from about 0.01 to about 100 mg/kg, from about 0.1 to about 100 mg/kg, from about 1 to about 100 mg/kg, from about 1 to about 10 mg/kg), every 4 to 120 hours, or according to the requirements of the particular drug. In some embodiments, it can be administered at a dose of at least about 0.01 ng/kg to about 100 mg/kg of body mass (e.g., about 10 ng/kg to about 50 mg/kg, about 20 ng/kg to about 10 mg/kg, about 0.1 ng/kg to about 20 ng/kg, about 3 ng/kg to about 10 ng/kg, or about 50 ng/kg to about 100 pg/kg) of body mass, although other dosages also may provide beneficial results. In some embodiments, the pharmaceutical composition is administered daily, bi daily, or weekly. The interrelationship of dosages for animals and humans (based on milligrams per meter squared of body surface) is described by Freireich et al., Cancer Chemother. Rep. 50, 219 (1966), hereby incorporated by reference in its entirety. Body surface area may be approximately determined from height and weight of the patient. See, e.g., Scientific Tables, Geigy Pharmaceuticals, Ardsley, N.Y., 537 (1970), hereby incorporated by reference in its entirety. In certain embodiments, the compositions are administered by oral administration or administration by injection. In some embodiments, the compositions are injected directly into an organ affected by TSC including the brain, a lung, the heart, and/or a kidney. The methods herein contemplate administration of an effective amount of compound or compound composition to achieve the desired or stated effect. In some embodiments, the pharmaceutical compositions of the present disclosure will be administered from about 1 to about 6 times per day or, alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Pharmaceutically acceptable carriers, adjuvants and vehicles that may be used in the compositions of the present disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat. Cyclodextrins such as γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, with each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges, or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

The compositions of the present disclosure may contain any conventional non-toxic pharmaceutically-acceptable carriers, adjuvants, or vehicles. In some cases, the pH of the formulation may be adjusted with pharmaceutically acceptable acids, bases, or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of the present disclosure may be orally administered in any orally acceptable dosage form including, but not limited to, capsules, tablets, emulsions and aqueous suspensions, dispersions, and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of the present disclosure may also be administered in the form of suppositories for rectal administration. These compositions can be prepared by mixing a compound of the present disclosure with a suitable non-irritating excipient that is solid at room temperature but liquid at the rectal temperature and therefore will melt in the rectum to release the active components. Such materials include, but are not limited to, cocoa butter, beeswax, and polyethylene glycols.

Topical administration of the compositions of the present disclosure is useful when the desired treatment involves areas or organs readily accessible by topical application. In some embodiments, topical administration further comprises topical application of sirolimus. For application topically to the skin, the composition should be formulated with a suitable ointment containing the active components suspended or dissolved in a carrier. Carriers for topical administration of the compounds of the present disclosure include, but are not limited to, mineral oil, liquid petroleum, white petroleum, propylene glycol, polyoxyethylene polyoxypropylene compound, emulsifying wax, and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol, and water. The compositions of the present disclosure may also be topically applied to the lower intestinal tract by rectal suppository formulation or in a suitable enema formulation.

In some embodiments, topical administration of the active compounds (e.g., TLR4 inhibitors, etc.) and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman, and Kanig, published by Lea and Febiger (1970) and in Remington's Pharmaceutical Sciences, 21st Edition (2005) published by Mack Publishing Company, which is incorporated herein by reference in its entirety.

Topically transdermal patches are also included in the present disclosure. Also within the present disclosure is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive; that is, it can allow for positioning of the adhesive (and the device to be adhered to the skin) against the skin by the application of pressure (e.g., pushing, rubbing) on the adhesive or device.

The compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having a TLR4 inhibitor and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). See Negrin et al., Biomaterials, 22 (2001): 563. Timed-release technology involving alternate delivery methods can also be used in the present disclosure. For example, timed-release formulations based on polymer technologies, sustained-release techniques and encapsulation techniques (e.g., polymeric, liposomal) can also be used for delivery of the compounds and compositions delineated herein.

The present disclosure will be further described in the following examples. It should be understood that these examples are for illustrative purposes only and are not to be construed as limiting the present disclosure in any manner. For example, one of ordinary skill will be able to exercise routine experimentation only, following the examples below, to ascertain compounds that have efficacy in treating or preventing TSC, or conditions associated with TSC.

### EXAMPLES

### Example 1

### qPCR of RNA Expression Encoding Inflammatory Proteins in Tsc1 Knockout Mice

A mouse model of TSC was created by the targeted disruption of the Tsc1 gene to identify the pathways that are affected by loss of Tsc1 or Tsc2 function. This model also allows for the investigation of the molecular and cellular response to putative TSC treatment (see, e.g., Example 2). Mice with one or two missing copies of Tsc1 allele were generated by the insertion of LoxP sites flanking exons 17 and 18 and excision of any floxed copy through activity of the CRE protein in astrocytes. Homozygous Tsc1 knockout mice (labeled in the figures as “CRE⁺; Tsc1^{flox/flox}”) develop TSC, while those heterozygous (labeled in the figures as “CRE⁺; Tsc1^{+/flox}”) do not in the absence of further manipulation. The CRE+ driver (which is expressed in astrocytes) targets only the foxed genes in the astrocytes, but has no effect on remaining portions of cells. Such Tsc1-GFAP-Cre knock-out mice with conditional inactivation of the Tsc1 gene predominantly in astrocytes were derived from those described in Uhlmann, E, et al., Ann Neurol 52 (2002): 285-296. Various markers of inflammation were measured in seven-week-old wild type (control) mice, Tsc1 heterozygous mice (Tsc1^{+/flox}), and Tsc1 homozygous knockout mice (Tsc1^{flox/flox}).

One hemisphere of fresh-frozen brain from each of the seven-week-old mice was homogenized 2×1 min at 25 Hz in 750 μL of QIAzol Lysis reagent (available from Qiagen, Valencia, Calif.) with TissueLyser (Qiagen, Valencia, Calif.) and 5 mm stainless steel beads (Qiagen, Valencia, Calif.). Disrupted samples were incubated at room temperature for five minutes. For RNA extraction, manufacturer protocol for RNeasy 96 Universal Tissue Kit (Qiagen, Valencia, Calif.) was followed. Briefly, 150 μL of chloroform (Sigma-Aldrich, St. Louis, Mo.) was added and samples were shaken vigorously for 15 seconds followed by 3 min incubation at room temperature. The aqueous phase was separated from the organic phase by centrifugation at 6,000×g (Beckman Coulter Avanti J-301) at 4° C. for 15 minutes. The aqueous phase was then transferred to a 96-well block, and total RNA was precipitated with equal volume of 70% ethanol. The precipitate was then transferred to an RNeasy 96-well plate followed by centrifuge at 6,000×g at room temperature for 4 min. Total RNA bound to column membranes was treated with RNase-Free DNAse set (Qiagen, Valencia, Calif.) for 30 minutes, followed by three washing steps with RW1 and RPE buffers (provided with RNeasy 96 Universal Tissue Kit). RNA was eluted with 20 μL RNase-Free water.

RNA was quantified using Nanodrop 8000. Total RNA (up to 1 μg of RNA) was reverse transcribed into cDNA with 3.2 μg random hexamers (Roche Applied Science, Indianapolis, Ind.), 1 mM dNTP (Roche Applied Science, Indianapolis, Ind.), 1× Transcriptor Reverse Transcription Reaction Buffer and 10U Transcriptor Reverse Transcriptase (Roche Applied Science, Indianapolis, Ind.) in 40 μL total volume mixture. The reactions were allowed to proceed at room temperature for 10 minutes, followed by 55° C. for 30 minutes and final inactivation at 85° C. for five minutes in a GeneAmp PCR Systems 97000 thermal cycler (Applied Biosystems, Foster City, Calif.). cDNA samples were diluted 10-fold with RNase-Free water for qPCR analysis.

All qPCR reagents and TaqMan Expression Assays were purchased from ThermoFisher Scientific. 5 μL of diluted cDNA was amplified with qPCR primer and probe sets in 1× TaqMan Fast Advanced Master Mix in 20 μL final reaction volume. Reactions were run on Applied Biosystems 7900HT Fast Block System with the following parameters: 95° C. for 20 seconds; 40 cycles of [95° C./3 s; 60° C./30 s]. Each cDNA sample for each assay was run in triplicates. The relative mRNA expression for each target was calculated using the 2^{-ΔΔCt} method with differences calculated with respect to the endogenous CANX gene.

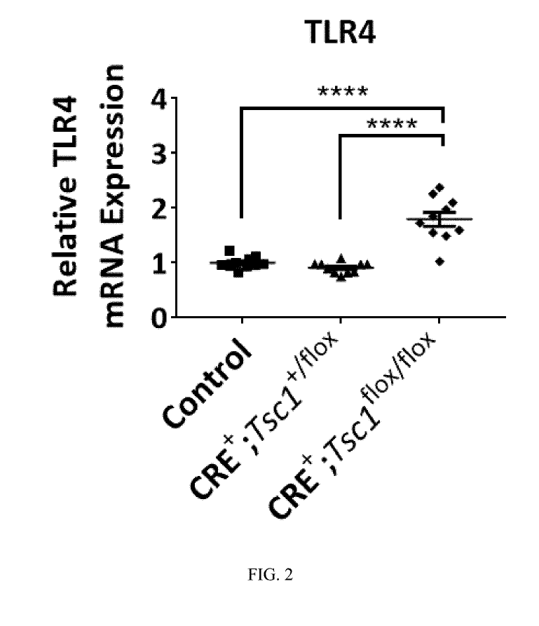

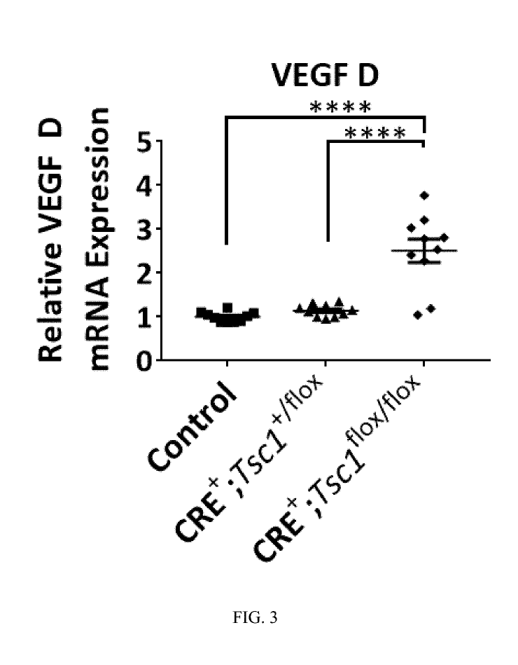

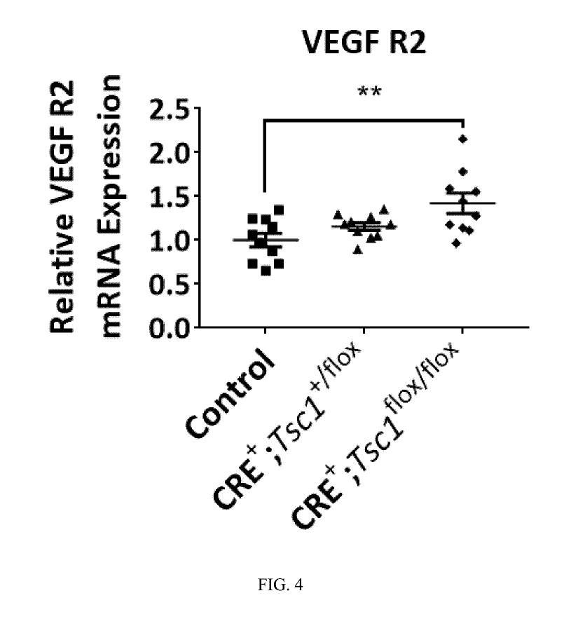

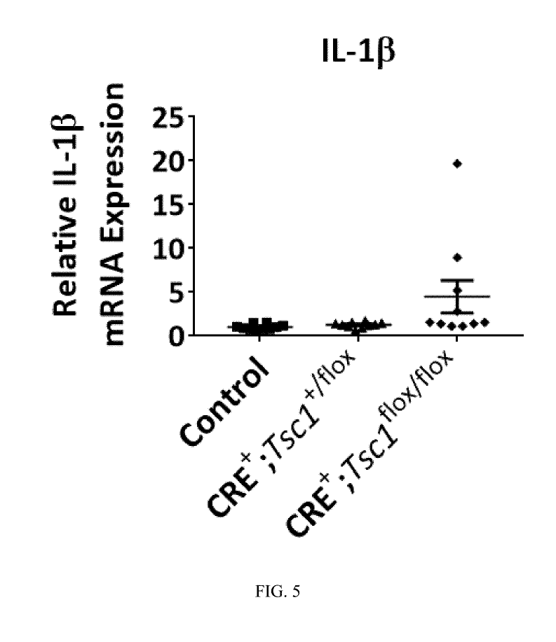

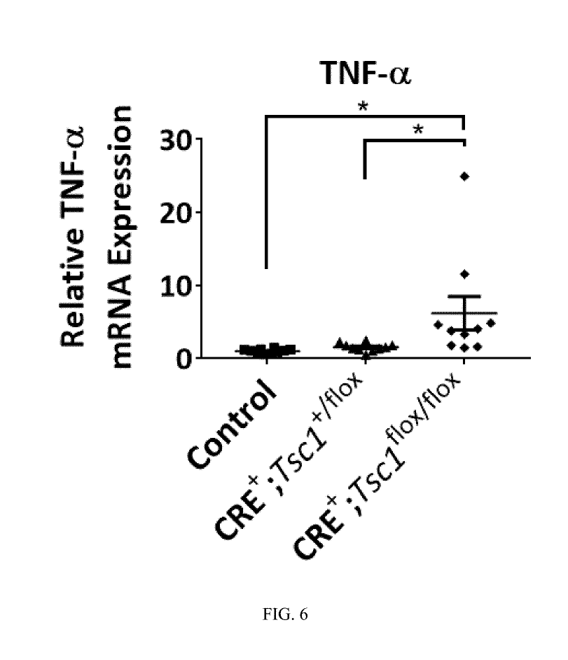

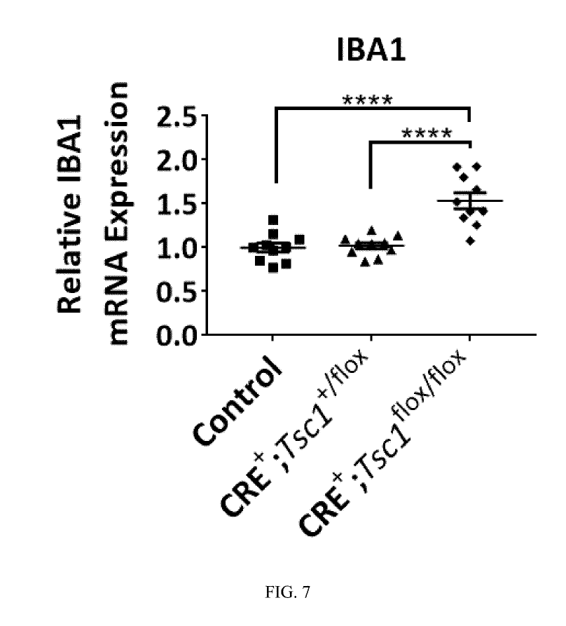

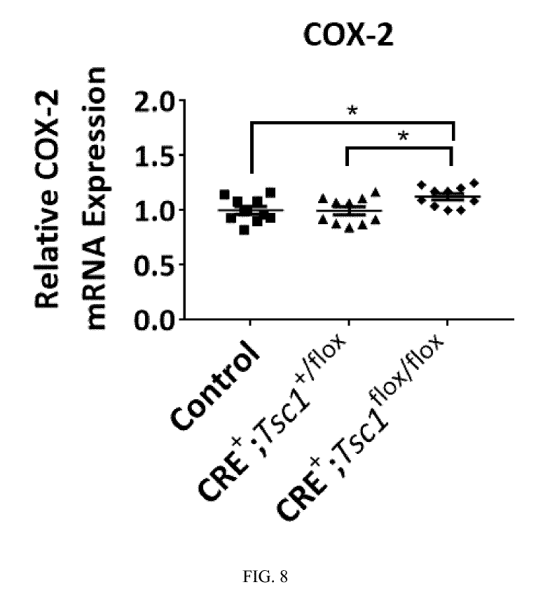

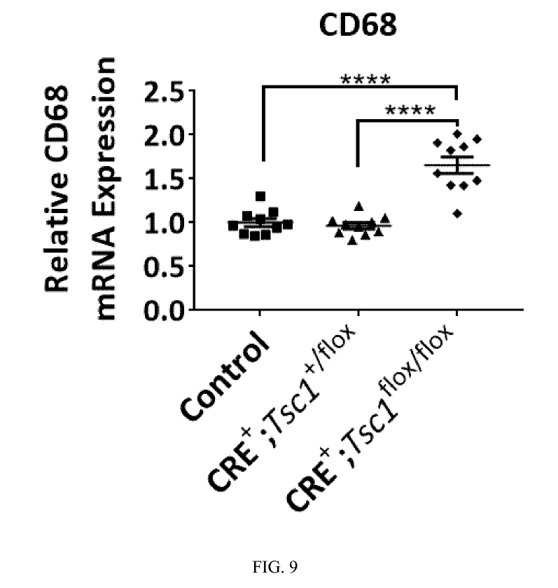

Several genes implicated in inflammation were shown to be upregulated in the homozygous knockout mice (CRE+; Tsc1^{flox/flox}) as compared to control (CRE+; Tsc1^{-/-}) and Tsc1 heterozygous mice (CRE+; Tsc1^{+/flox}). FIGS. 2-9 illustrate the results of the qPCR analysis. Normalized target expression is expressed relative to the average control. As can be seen, heterozygous mice have no increase in expression of these inflammation linked genes, while Tsc1 homozygous knockout mice show upregulation. TLR4 and its downstream targets in the inflammation signaling pathway (VEGF D, VEGF R2, IL-1β, TNF-α, IBA1, COX-2, CD68) are each upregulated in the TSC1 homozygous knockout mice. Moreover, the upregulation of NR2B in TSC human cortical tubers, as disclosed in Talos, D. et al., Ann. Neural. 63 (2008): 454-465, may due to its position downstream from TLR4 in the pathway. The upregulation of each measured target is statistically significant over control as indicated (* indicates p<0.05, ** indicates p<0.01, *** indicates p<0.001, and **** indicates p<0.0001).

### Example 2

### qPCR of RNA Expression Encoding Inflammatory Proteins in Tsc1 Knockout Mice Treated with Rapamycin

Tsc1 homozygous knockout mice were developed as described in Example 1. At approximately three weeks (day 22-25) of age, 33 mice were implanted with electrodes for EEG measurements yielding a group of 30 mice for study. Daily intraperitoneal injections of a formulation were performed on each mouse starting at 21 days of age and lasted for four weeks (7 days per week for 4 weeks totaling 28 doses per mouse). Mice were either given vehicle alone for days 21-48 of age (“Vehicle”), vehicle with rapamycin for days 21-48 of age (”Long Rapamycin“), or vehicle alone for days 21-34 followed by vehicle with rapamycin for days 35-48 of age (“Short Rapamycin”). The vehicle was 5% tween 80 (v/v), 5% PEG 400 (v/v), 4% ethanol (v/v) in saline (0.9% sterile saline USP). Rapamycin was dosed per injection at 3 mg (rapamycin)/kg (mouse); 5 mL (formulation)/kg (mouse) IP.

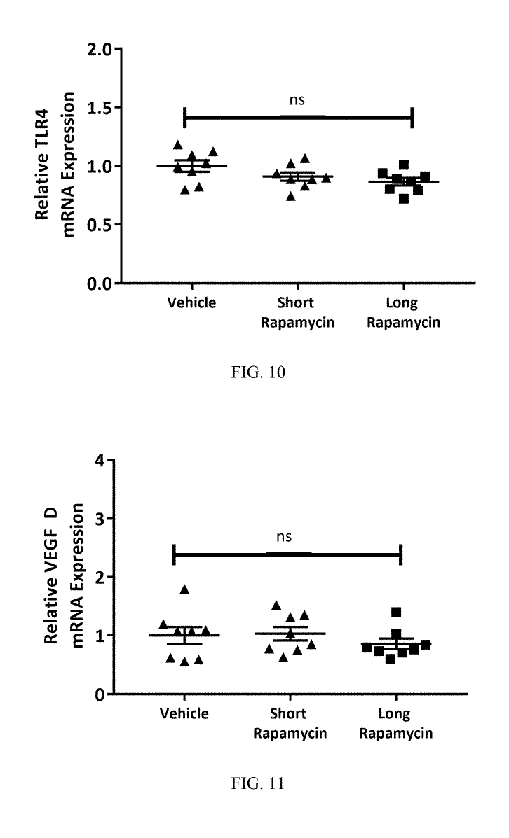

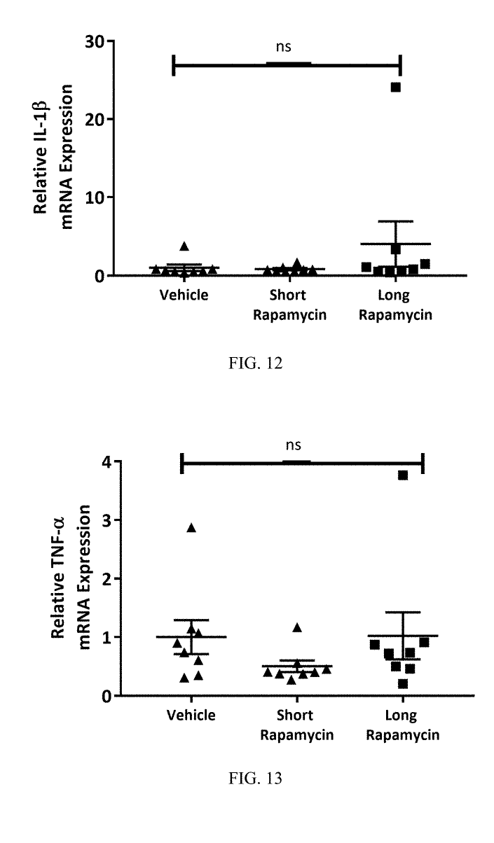

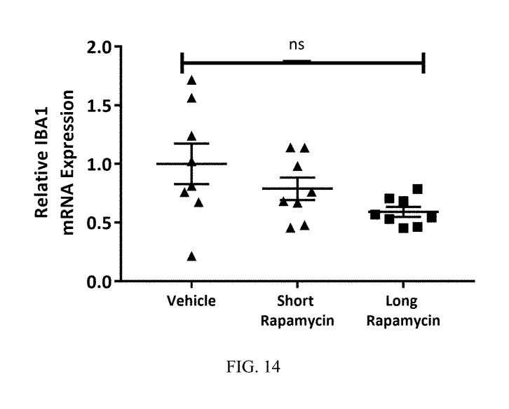

At the age of seven weeks (day 49) of age, qPCR analysis of several cytokines, mediators, and receptors in the brain of treated mice was performed as in Example 1. FIGS. 10-14 illustrate the results of the qPCR analysis. Normalized target expression is expressed relative to the average control. As can be seen, several cytokines and receptors implicated in the TLR4 pathway, including the TLR4 receptor itself, are not affected by the mTOR inhibition treatment with rapamycin. However, rapamycin is known to treat TSC. Accordingly, the use of a TLR4 inhibitor in combination with an mTOR inhibitor may provide an unexpectedly increased therapeutic benefit in the treatment or prophylaxis of TSC and/or conditions associated with TSC.

### Example 3

### Measurements on Tsc1 Knockout Mice Treated with TLR4 Inhibitors

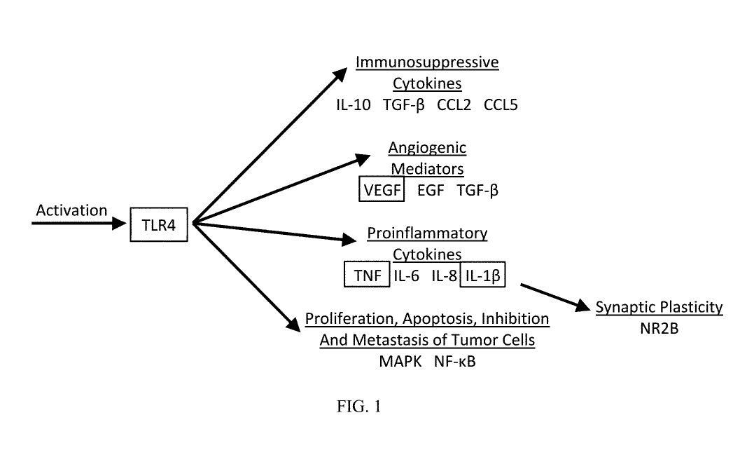

CRE⁺; Tsc1^{flox/flox} knockout mice may be treated with a TLR4 inhibitor for an amount of time prior to analysis. qPCR analysis of brain tissue is expected that a downregulation of the downstream inflammatory effects TLR4 (see FIG. 1) will occur. Additionally, it is believed that EEG measurements which identify seizure frequency and duration of mice will show that administration of TLR4 inhibitors prior to the manifestation of seizures in Tsc1 homozygous knockout mice can prevent the onset of seizures. Also, EEG measurements may show that administration of TLR4 inhibitors after the manifestation of seizures in Tsc1 homozygous knockout mice can reduce the frequency and/or duration of seizures or remove seizure occurrence altogether.

Unless otherwise specified, all references including patent applications and publications cited herein are incorporated herein by reference and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method for the treatment or prophylaxis of tuberous sclerosis complex (“TSC”) in a patient in need thereof comprising administering a therapeutically effective amount of a pharmaceutical composition comprising a TLR4 inhibitor in combination with one or more pharmaceutical carriers, excipients, or diluents to said patient.

2. The method according to claim 1, wherein said one or more TLR4 inhibitors is selected from Eritoran, Ibudilast, Rhodobacter sphaeroides LPS (Lps-Rs)37, IAXO-102, VIPER (from vaccinia virus protein A46), Cyanobacterial LPS (Cyp)38, Bartonella quintana lipopolysaccharide, natural and unnatural enantiomers of naloxone, amitriptyline, cyclobenzaprine, ketotifen, imipramine, mianserin, naltrexone, propentofylline, and tapentadol, or pharmaceutically acceptable salts, or prodrugs thereof.

3. The method according to claim 1, wherein said TLR4 inhibitor is an anti TLR4 antibody (e.g., a monoclonal antibody).

4. The method according to claim 1, wherein said pharmaceutical composition further comprises an NSAID, a mTOR inhibitor, an I1-1β inhibitor, a CXCL10 inhibitor, and combinations thereof.

5. The method according to claim 1, wherein said pharmaceutical composition comprises from about 1 μg to about 1000 mg of said one or more TLR4 inhibitors.

6. The method according to claim 1, wherein said therapeutically effective amount is from about 1 μg to about 500 mg of said one or more TLR4 inhibitors per kg of body weight of said patient.

7. The method according to claim 1, wherein said administration occurs daily.

8. The method according to claim 1, wherein said pharmaceutical composition is administered orally.

9. The method according to claim 1, wherein said pharmaceutically composition is in the form of a tablet or a capsule.

10. The method according to claim 1, wherein said pharmaceutical composition is administered parenterally.

11. The method according to claim 1, wherein said pharmaceutical composition is administered intraperitoneally, intravenously, intramuscularly, intra-arteriole, intradermally, subcutaneously, intraventricularly, or intracranially.

12. The method according to claim 1, wherein said administration occurs before a patient has been diagnosed with one or more TSC-associated conditions.

13. The method according to claim 1, wherein said administration occurs after a patient has been diagnosed with one or more TSC-associated conditions.

14. The method according to claim 1, wherein said administration occurs after a patient has been definitely diagnosed with one or more TSC-associated conditions.

15. The method according to claim 1, wherein said administration occurs after a patient has been possibly diagnosed with one or more TSC-associated conditions.

16. A method for the treatment or prophylaxis of one or more TSC-associated conditions to a patient in need thereof comprising administering to said patient a therapeutically effective amount of a pharmaceutical composition comprising a TLR4 inhibitor.

17. The method according to claim 16, wherein said TLR4 inhibitor is selected from Eritoran, Ibudilast, Rhodobacter sphaeroides LPS (Lps-Rs)37, IAXO-102, VIPER (from vaccinia virus protein A46), Cyanobacterial LPS (Cyp)38, Bartonella quintana lipopolysaccharide, natural and unnatural enantiomers of naloxone, amitriptyline, cyclobenzaprine, ketotifen, imipramine, mianserin, naltrexone, propentofylline, and tapentadol, or pharmaceutically acceptable salts, or prodrugs thereof.

18. The method according to claim 16, wherein said pharmaceutical composition further comprises a NSAID, a mTOR inhibitor, an I1-1β inhibitor, a CXCL10 inhibitor, and combinations thereof.

19. The method according to claim 16, wherein said pharmaceutical composition comprises about 1 μg to about 1000 mg of said TLR4 inhibitor.

20. The method according to claim 16, wherein said pharmaceutical composition is administered orally.

21-129. (canceled)
